(19) United States
(12) Patent Application Publication
(10) Pub. No.: US 2009/0062911 A1
(43) Pub. Date: Mar. 5, 2009

(54) MULTIZONAL LENS WITH EXTENDED DEPTH OF FOCUS

(75) Inventor: Theophilus Bogaert, Groningen (NL)

Correspondence Address: ADVANCED MEDICAL OPTICS, INC., 1700 E. ST. ANDREW PLACE, SANTA ANA, CA, 92705, US

(73) Assignee: AMO GRONINGEN BV, Groningen (NL)

(21) Appl. No.: 11/845,682

(22) Filed: Aug. 27, 2007

(51) Int. Cl. A61F 2/16

(52) U.S. Cl. 623/6.27

(57) ABSTRACT

An intraocular lens for providing enhanced vision includes an optic with a clear aperture over which light may be focused onto or near the retina of an eye. The optic includes an anterior surface and an opposing posterior surface, the surfaces disposed about an optical axis. The optic includes a central zone and an outer zone disposed about the central zone. The central zone comprises a plurality of optical powers that progressively vary between a first optical power at a center of the central zone and a second optical power at a periphery of the central zone, wherein the absolute value of the difference between the first optical power and the second optical power being between predetermined values. The outer zone comprises a third optical power and may also have a negative spherical aberration. The optic typically has a variation in optical power over the entire clear aperture that is less than a predetermined amount.

### FIELD OF THE INVENTION

This invention relates to devices and method for enhancing the vision of a subject and, more particularly, to multi-zonal ophthalmic lenses and method of malting that correct aberrations and provide an extended depth of focus.

### BACKGROUND OF THE INVENTION

Intraocular lenses and other ophthalmic devices are used to restore and correct vision. For example, monofocal intraocular lenses may be used to replace the natural lens of an eye that has developed cataracts. The simplest types of lenses to fabricate are generally spherical lenses in which both surfaces of the lens have a spherical profile. More recently, aspheric lenses have been used to replace or supplement the eye's natural lens. Such aspheric lenses may be used to at least partially correct for aberrations that are produced by spherical surfaces and/or aberrations produced by the eye itself (e.g., positive spherical aberrations produced by the cornea of most human eyes). Examples of such lens designs are described in U.S. Pat. Nos. 6,609,793 and 7,137,702, which are herein incorporated by reference in their entirety. Lenses may also be configured to correct for chromatic aberrations inherent in most refractive lenses, for example, through the use of diffractive phase plates (e.g., U.S. Pat. Nos. 4,637,697, 5,121,979, and 6,830,332 and U.S. Patent Application Number 2004/0156014 and 2006/0098163, all of which are also herein incorporated by reference in their entirety).

When spherical intraocular lenses are used, a practitioner may select a lens power based on a so called “hyperfocal distance”, which may make a subject slightly myopic. One advantage of this choice is an increased likelihood that the subject will have spectacle free vision for at least one distance (e.g., if preoperative measurements result in an intraocular lens that is too strong, then the lens will at least provide near or intermediate vision without the use of spectacles or contact lenses). Another benefit of this approach is that lens power selection based on the hyperfocal distance generally provides for the greatest range of distances over which objects at different distances will be reasonably clear to the subject, without the use of spectacles or contact lenses. The increased focal range provided by choosing the hyperfocal distance results in a type of pseudo-accommodation that can resemble the vision provided by the eye's natural lens prior to the onset of presbyopia.

One potential drawback to selecting the optical power of an intraocular lens to correspond to the hyperfocal distance is that visual acuity for nighttime driving may be reduced, since the best lens performance has been set for objects located between the hyperfocal distance and optical infinity. By contrast, most of the objects within the field of view under these conditions are at optical infinity and, therefore, slightly defocused. Since the pupil is fully dilated under these conditions, spherical aberrations may further reduce visual acuity. Spherical aberrations may be reduced by using aspheric lens surfaces that are configured to correct or compensate for spherical aberrations of the lens and/or cornea.

Regardless of in-focus condition selected (e.g., at optical infinity or at the hyperfocal distance), aspheric lens surfaces serve to provide an improved visual outcome. This is because, as compared to a substantially equivalent spherical lens, aspheric lenses generally provide better visual acuity or MTF performance at all distances and not simply at the distance corresponding to the in-focus condition wherein light is focused on the surface of the retina. Thus, while an aspheric lens with a power selected for the hyperfocal distance generally provides better nighttime driving vision than is possible with a spherical lens, the visual acuity will still be at least somewhat reduced as compared that when the power of the lens is selected to provide emmetropia.

Accordingly, improved designs in monofocal ophthalmic lenses are needed that will provide both increased visual acuity under nighttime driving conditions, while also providing the relatively large depth of focus under other lighting conditions that is possible by selecting a lens power based on the hyperfocal distance.

### SUMMARY OF THE INVENTION

Embodiments of the present invention are generally directed to devices and methods for providing an eye with enhanced visual acuity under certain visual and/or lighting conditions (e.g., by reducing spherical aberrations or other aberrations of the lens and/or eye under typical nighttime driving conditions) while simultaneously providing a relatively large depth of field or depth of focus under other lighting conditions as compared to traditional spherical and/or aspheric lenses (e.g., under typical indoor or outdoor lighting conditions or under typical reading conditions). Exemplary embodiments of the invention presented herein are generally directed to intraocular lenses; however, embodiments of the invention may also be extended to other types of ophthalmic lenses and devices, such as corneal inlays or onlays, phakic lenses, laser vision correction (e.g., LASIK and PRK procedures), contact lenses, and the like.

One aspect of the present invention involves an ophthalmic device, such as an intraocular lens, comprising an optic having a variation in optical power over the entire clear aperture that is less than a predetermined amount that is relatively small compared to the add power of a typical refractive or diffractive multifocal intraocular lens (e.g., less than about 2 Diopters or 1.5 Diopters). The optic has a clear aperture over which incident light is focused onto the retina of an eye, an anterior surface, and an opposing posterior surface, the surfaces disposed about an optical axis. The optic further comprises a central zone having a plurality of optical powers that progressively vary between a first optical power at a center of the central zone and a second optical power at a periphery of the central zone, wherein the absolute value of the difference between the first optical power and the second optical power is within a relatively small range compared to the add power of a typical refractive or diffractive multifocal intraocular lens (e.g., between about 0.25 Diopter and about 2 Diopters). The ophthalmic devices also comprises an outer zone disposed about the central zone, the outer zone comprising a third optical power and optionally an optical aberration to compensate or reduce a similar aberration of the cornea or eye of a subject. The optical aberration may be a chromatic aberration or a monochromatic aberration such as a spherical aberration, a coma, or an astigmatism.

Another aspect of the present invention involves a method of making an intraocular lens or other ophthalmic device, the method comprising forming an anterior surface and an opposing posterior surface, the surfaces being disposed about an optical axis and providing a clear aperture. The method also comprises forming a central zone comprising a plurality of optical powers that progressively vary between a first optical power at a center of the central zone and a second optical power at a periphery of the central zone. The method further comprises forming outer zone disposed about the central zone, the outer zone comprising a third optical power and an optionally an optical aberration. The optic has a variation in optical power over the entire clear aperture that is less than a predetermined amount that is relatively small compared to the add power of a typical multifocal intraocular lens (e.g., less than 3 Diopters or 4 Diopters).

Each and every feature described herein, and each and every combination of two or more of such features, is included within the scope of the present invention, provided that the features included in such a combination are not mutually inconsistent.

### BRIEF DESCRIPTION OF THE DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a side view of an intraocular lens comprising spherical surfaces and disposed within an eye.

FIG. 2 is a side view of the intraocular lens of FIG. 1 in which an object or point source is disposed nearer the eye.

FIG. 3 is a side view of an intraocular lens comprising at least one aspheric surface configured to reduce a spherical aberration of the eye.

FIG. 4A is a side view of an intraocular lens comprising a central zone and a peripheral zone according to an embodiment of the present invention.

FIG. 4B is a front view of the intraocular lens of FIG. 4A.

FIG. 5 is a magnified side view of the intraocular lens of FIG. 4A particularly showing the central zone of the intraocular lens.

FIG. 6 is a is another embodiment of a central zone for use in an intraocular lens according to embodiments of the present invention.

### DETAILED DESCRIPTION OF THE DRAWINGS

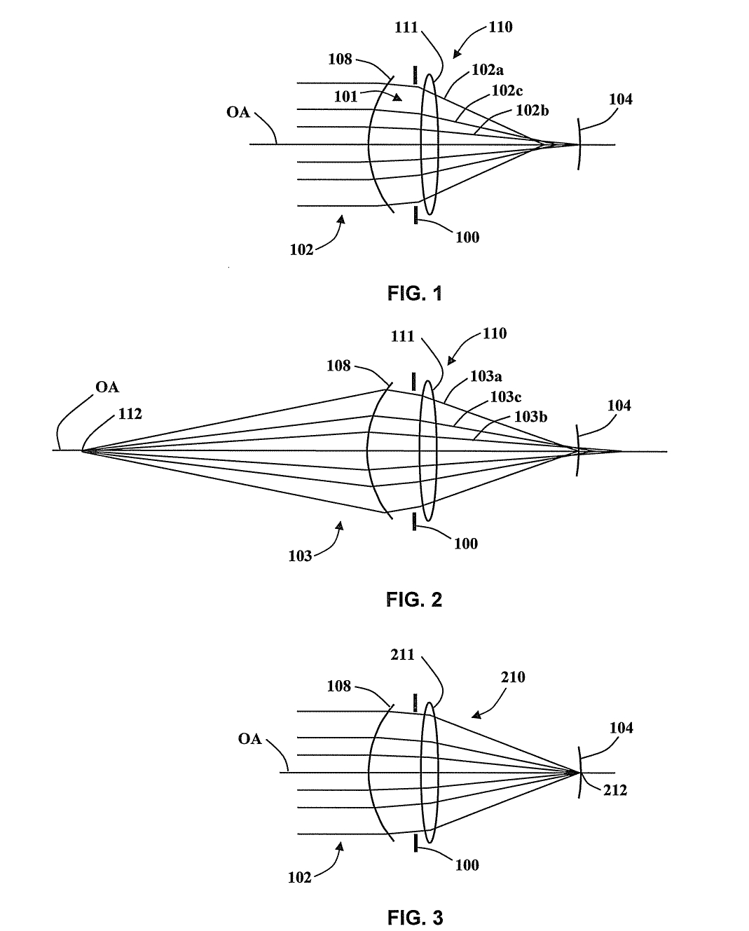

Referring to FIG. 1, an eye is generally disposed about an optical axis OA and comprises an iris 100 forming a pupil 101 through which a plurality of rays 102 from a distant object or point source enter the pupil 101 and are generally focused onto a retina 104 by a cornea 108 and a monofocal intraocular lens 110 comprising an spherical optic 111 with anterior and posterior surfaces that are spherical. For simplicity, other portions and elements of the eye apart from those shown in FIG. 1 have been left out. The combination of the spherical surfaces of the intraocular lens 110 and the corneal surface shape cause peripheral ray 102a to focus closer to the intraocular lens 110 than does inner or paraxial ray 102b. The focal length of the intraocular lens 110 may be represented by an intermediate ray 102c that focuses on the optical axis OA at a location intermediate to the foci of the rays 102a, b. The ray 102c may correspond to a ray that intercepts the optical axis OA at a plane located at the so-called “circle of least” confusion, although other criteria may be used for determining and defining the focal length of the intraocular lens 110. For purposes of this disclosure, the focal length of an intraocular lens (or a region or zone thereof) is the reciprocal of an average optical power over the intraocular lens (or region or zone), where the optical power is expressed in units of Diopters or m⁻¹.

The difference in focal location of the rays 102a-c illustrated in FIG. 1 is due, at least in part, to a variation in optical power of the optic 111 with radius from the optical axis OA that results from the use of anterior and posterior lens surfaces that are spherical. This difference in focal location is referred to as a spherical aberration. Since the peripheral ray 102a comes to focus closer to the optic 111 than the paraxial ray 102b, the spherical aberration is said to be a positive spherical aberration. A negative spherical aberration would occur if the peripheral ray 102a were to come to focus farther from the optic 111 than the paraxial ray 102b. The spherical aberration illustrated in FIG. 1 is the result of the combined spherical aberrations optic 111 and the cornea 108.

When the optical power of an optic or optical system varies continuously with distance from the optical axis OA (e.g., as illustrated in FIG. 1), the range of resulting foci along the optical axis OA may be related to an increased depth of focus or depth of field (DOF) of the optic. In the case of the intraocular lens 110, the optical power of the lens is selected so that the paraxial ray 102b is focused onto the retina 104. Thus, at least some light from the distant point source represented in FIG. 1 is focused onto the retina 104 so that the intraocular lens 110 provides distant vision that is clinically equivalent to, or at least similar to, that provided if the intraocular lens 110 were configured to focus the ray 102c onto the retina 104.

With further reference to FIG. 2, an advantage of the arrangement illustrated in FIG. 1 is that an object or point source 112 located at a so-called hyperfocal distance from the eye is also just focused by the optic 111 and cornea 108. As illustrated, a plurality of rays 103 from object source 112 are focused by the cornea 108 and the intraocular lens 110 (e.g., rays 103a-c). The peripheral ray 103a is just focused onto the retina 104, so that an object or point source disposed at the hyperfocal distance is perceived with similar visual acuity as a distant point source at optical infinity. Such an arrangement allows the spherical intraocular lens 110 and/or cornea 108 to provide a pseudo-accommodation in which both distant objects and objects located at intermediate distances (e.g., at or near the hyperfocal distance) are suitably resolved by the eye. As used herein, the term “hyperfocal distance” means a distance from a healthy, emmetropic eye, at which an add power of 0.5 Diopters in the spectacle plane provides visual acuity at least 20/20, based on the standard Snellen test for visual acuity. For example, in a human eye with an axial length (AL) of 25 mm, the hyperfocal distance is approximately 2.5 meters from the eye. As used herein, the term “emmetropic eye” means an eye having a visual acuity for distant vision of at least 20/20, based on the standard Snellen test for visual acuity. As used herein, the term “emmetropic vision” means vision which provides a visual acuity for distant object of at least 20/20.

Referring now to FIG. 3, an aspheric intraocular lens 210 is illustrated that comprises an optic 211 in which at least one of the surfaces is aspheric, such that all the rays 102 from a distant object or point source come to a common focus 212 on the surface of the retina 104. The surfaces of the optic 211 may be configured such that the optic 211 has a negative spherical aberration that is selected to compensate, reduce, or cancel a positive spherical aberration produced by a spherical surface of the optic 211 and/or by a spherical aberration of the cornea 108. Additionally or alternatively, the intraocular lens 210 may be configured to comprise another monochromatic aberration that compensates, reduces, or cancels a substantially opposite aberration of the cornea 108 and/or a remaining spherical surface of the optic 211. As discussed in U.S. Pat. No. 6,609,793, the cornea 108 may represent an average model cornea (e.g., based on a population having a common characteristic). In such instances, the surfaces of the optic 211 may have a negative spherical aberration that partially or completely compensates for a spherical aberration of the cornea 108. The population may, for example, represent patients that are candidates for a cataract surgery, patients within a certain age group, and/or having a common or similar aberration or set of aberrations.

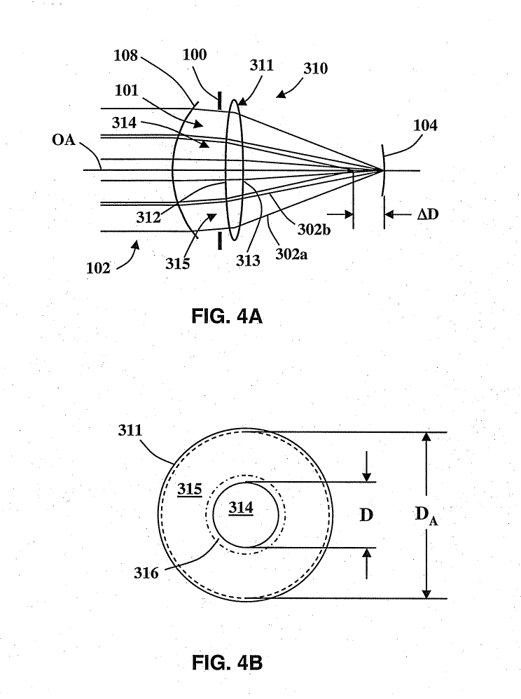

Referring to FIGS. 4A and 4B, an intraocular lens 310 according to an embodiment of the present invention is disposed within an eye and comprises an optic 311. The optic 311 is configured to provide an extended DOF (e.g., similar to that illustrated for the intraocular lens 110) under certain lighting conditions, while also providing enhanced visual acuity (e.g., similar to that produced by an aspheric intraocular lens illustrated for the intraocular lens 210) for other lighting conditions. The optic 311 has a clear aperture over which light incident thereon is focus onto the retina 104. The optic 311 includes an anterior surface 312 and an opposing posterior surface 313, the surfaces 312, 313 being disposed about an optical axis OA.

As used herein, the term “clear aperture” means the opening of a lens or optic that restricts the extent of a bundle of light rays from a distant source that can imaged or focused by the lens or optic. The clear aperture is usually circular and specified by its diameter. Thus, the clear aperture represents the full extent of the lens or optic usable in forming the conjugate image of an object or in focusing light from a distant point source to a single focus or to a plurality of predetermined foci, in the case of a multifocal optic or lens. It will be appreciated that the term “clear aperture” does not denote or imply a particular clarity or transmissivity of an optic or lens. For example, an optic may have a clear aperture that is approximately equal to the diameter of the optic, irrespective of whether or not a dye is used to reduce the transmission of light.

In the illustrated embodiment, the clear aperture has a diameter DA that is substantially equal to the diameter of the optic 311. As illustrated in FIG. 4B, the diameter DA may be slightly smaller than the outer diameter of the optic 311, for example, due to the presence of a peripheral edges that includes rounded corners not useful in focusing light or forming an image on the retina 104. In some embodiment, the peripheral edge is configured to reduce scatter from light sources at the periphery of the field of view of a subject into which the lens 210 is implanted. Thus, the glare reducing peripheral edge may slightly reduces the clear aperture of the optic 311.

The optic 311 comprises a central zone 314 and an outer zone 315 disposed about the central zone 314. The central zone 314 includes a plurality of optical powers that progressively vary between a first optical power P1 at or near a center of the central zone 314 and a second optical power P2 at or near a periphery of the central zone 314. The absolute value of the difference between the first optical power P1 and the second optical power P2 (e.g., |P2−P1|) is generally less than the add power of a typical multifocal intraocular lens (e.g., less than about 3 or 4 Diopters). For example, the absolute difference between P1 and P2 is generally between about 0.1 Diopter and about 1.5 Diopter or between about 0.25 Diopter and about 1 Diopter.

In certain embodiments, the central zone 314 may comprise a plurality of distinct powers that are produced through the use of a refractive or diffractive surface profile. For example, the central zone may comprise a diffractive grating or phase plate that produces two distinct foci. In general, the difference in optical power between the two foci is relatively small (e.g., less than or equal to 1 Diopter, 1.5 Diopters, or about 2 Diopters), although larger Diopter differences may be incorporated. In some embodiments, a relatively small difference in optical power between the two foci may be used to provide an extended depth of focus, for example, as disclosed in co-pending U.S. Provisional Patent Application No. 60/968,250, which is herein incorporated by reference in its entirety.

The outer zone 315 of optic 311 comprises a third optical power P3 that may be equal to P1 or P2, between P1 and P2, or outside the range between P1 and P2. Either or both of the zones 314, 315 may include a monochromatic and/or chromatic aberration that is selected to improve vision when the pupil 101 is relatively large (e.g., under low light conditions or typical room light). For example, at least one of the surfaces 312, 313 in the vicinity of the outer zone 315 may have a negative spherical aberration that at least partially compensates for a positive spherical aberration of the cornea and/or for a positive spherical aberration of one or both of the surfaces of the optic 311. The outer zone generally has an outer diameter that is equal to the outer diameter of the clear aperture. Alternatively, the outer zone 315 may be surrounded by an additional zone (not shown) having a predetermined radial profile that provides a particular optical characteristic.

The zones 314, 315 are configured such that the optic 311 has a variation in optical power over the entire clear aperture that is less than about 1.5 Diopters or less that about 1 Diopter. The total variation in optical power over the entire clear aperture may be selected in accordance with specific design parameters such as the range of pseudo-accommodation to be provided, the required visual acuity at one or more specific object distance, the zone diameters, the pupil size under certain lighting conditions, the expected variation in pupil size, a desired mixture of near, intermediate, and/or distant vision for one or more pupil sizes, and the like.

The optic 311 in the illustrated embodiment is circular; however, other shapes may be used, for example, to enhance the insertion characteristics of the intraocular lens 310 into the eye through a small incision. Also, at least one of the zones 314, 315 may comprise a cylinder power, for example, to correct for an astigmatism of the eye. While not illustrated in the FIG. 4A or 4B, it will be appreciated that the intraocular lens 310 may generally comprise other features and elements such as edge features for reducing glare and/or reducing PCO, haptics for centering the intraocular lens 310 within the eye, and/or a positioning structure for providing accommodative axial motion and/or deformation of the optic 311. The optic 311 may be a single optic or part of a lens system, for example, one of the lenses of a two lens accommodating intraocular lens. In addition, the intraocular lens 310 may be configured to attenuate light over a band of wavelengths light outside a band of wavelengths. In such embodiments the intraocular lens 310 or the optic 311 may incorporate one or more dyes or other substances or devices for selectively blocking incident radiation, for example, to selectively blocking UV radiation or light in the violet or blue bands of the visible spectrum.

The intraocular lenses 310 may be fabricated with optical powers that vary from about 10 Diopters to about 30 Diopters in increments of about 0.5 Diopters. In some embodiments, intraocular lenses 310 may be produced that vary from about zero Diopters to about 40 Diopters or more. Alternatively or additionally, intraocular lenses 310 may be produced that have a negative optical power, for example that is within a range of less than about zero Diopters to greater than about −20 Diopters or less.

The intraocular lens 310 may generally be constructed of any of the various types of material known in the art. For example, the intraocular lens 310 may be a foldable lens made of at least one of the materials commonly used for resiliently deformable or foldable optics, such as silicone polymeric materials, acrylic polymeric materials, hydrogel-forming polymeric materials (e.g., polyhydroxyethylmethacrylate, polyphosphazenes, polyurethanes, and mixtures thereof), and the like. Other advanced formulations of silicone, acrylic, or mixtures thereof are also anticipated. Selection parameters for suitable lens materials are well known to those of skill in the art. See, for example, David J. Apple, et al., Intraocular Lenses: Evolution, Design, Complications, and Pathology, (1989) William & Wilkins, which is herein incorporated by reference. The lens material may be selected to have a relatively high refractive index, and thus provide a relatively thin optic, for example, having a center thickness in the range of about 150 microns to about 1000 microns, depending on the material and the optical power of the lens. At least portions of the intraocular lens 310, for example one or more haptics or fixation members thereof, may be constructed of a more rigid material including such polymeric materials as polypropylene, polymethylmethacrylate PMMA, polycarbonates, polyamides, polyimides, polyacrylates, 2-hydroxymethylmethacrylate, poly (vinylidene fluoride), polytetrafluoroethylene and the like; and metals such as stainless steel, platinum, titanium, tantalum, shape-memory alloys, e.g., nitinol, and the like. In some embodiments, the optic and haptic portions of the intraocular lens 310 are integrally formed of a single common material.

As illustrated in FIG. 4A, a pair of rays 302a, 302b from the light 102 impinge upon the outer zone 315 near the outer periphery thereof and near the central zone 314, respectively. At least one of the surfaces 312, 313 in the region of the outer zone 315 is preferably aspheric in shape, such that light passing through the outer zone 315 is focused to substantially a single point or focus (e.g., to within a circle about the size of an Airy disk defining a diffraction limit of the zone 315). For example, the outer zone 315 may be configured to have at least some of the features and/or functions describe above with regards to the optic 211 illustrated in FIG. 3. In this regard, the outer zone 315 may comprise a monochromatic aberration, such as a spherical aberration, that corrects or at least partially compensates for an aberration of the eye (e.g., a spherical aberration introduced by the cornea 108). Additionally or alternatively, the outer zone 315 may incorporate a chromatic aberration, for example, through the use of a diffractive grating or phase plate on one of the lens surfaces. The aberration of the outer zone 315 may be selected to correct the aberrations of an individual cornea, in which case the intraocular lens 310 may be a custom intraocular lens. Alternatively, the intraocular lens 310 may be selected from a plurality intraocular lenses or optic portions having the same optical power, but differing amounts of spherical aberration. Alternatively, the aberration of the outer zone 315 may be selected to compensate for an aberration of a cornea that is part of an eye model and/or that represents an average cornea based on a particular population (e.g., an average spherical aberration for a population of people of a particular age group or that are likely candidates of a particular surgical procedure). The outer zone 315 of the intraocular lens 310 may be configured to have a surface sag profile that varies according to the relation:

$$\frac{{cr}^{2}}{1 + \sqrt{1 - ( 1 + k ) {c}^{2} {r}^{2}}} + {a}_{4} {r}^{4} + {a}_{6} {r}^{6} + …$$

wherein a₂, a₄ . . . are constants, c is a base curvature of the surface portion, k is a conic constant, and r is the radial distance from the optical axis OA.

The aberration of the outer zone 315 may be selected to completely or substantially completely compensate for a spherical aberration of a cornea or eye. Alternatively, the aberration of the outer zone 315 may be selected to only partially compensate for (or over compensate for) the spherical aberration or other aberration of the cornea or eye. In this regard, it may be advantageous in certain embodiments to select the aberration of the outer zone 315 to leave a residual aberration when combined with a cornea, for example, as discussed in U.S. Patent Application Number 2006/0244904, which is herein incorporated by reference in its entirety. For instance, the intraocular lens 310 may comprise an outer zone 315 that has an optical power that is about 20 Diopters and a negative spherical aberration that partially correct a positive spherical aberration of the cornea, wherein the outer zone 315 has a negative spherical aberration that is between about −0.19 and about −0.202 microns, or that is about −0.156 microns.

The eye may have a residual aberration that is essentially zero or is greater than zero (e.g., a residual aberration of about +0.14 microns or between about +0.006 microns and about +0.090 microns has been reported as potentially beneficial, for example, when placed in an eye or an eye model with a corneal spherical aberration of about 0.327 microns). In other embodiments, the intraocular lens 310 is configured with an outer zone 315 in which the optical power at the periphery of the zone is about 0.5 to about 0.75 Diopters less than the optical power at or near the boarder between the zones 314, 315.

As discussed above, the central zone 314 has an optical power that ranges from P1 at or near the center to P2 at or near the periphery of the zone, while the outer zone 315 has a power P3. In certain embodiments, the first optical power P1 and/or the third optical power P3 is less than the second optical power P2 by an amount that is less than or equal to about 1.5 or 2 Diopters, preferably less than or equal to about 1.0 Diopter, and in some cases less than or equal to about 0.5 Diopters. In some embodiments, the variation in optical power over the entire clear aperture (e.g., within and between the zones 314, 315) is less than or equal to about 0.5 Diopter plus the variation in optical power produces by the spherical aberrations of a spherical optic having a nominal optical power equal to that of the third optical power P3.

In the illustrated embodiment, the difference between the second optical power P2 of the central zone 314 and the first and/or third optical powers P1, P3 represents an add power ΔD, where the add power ΔD is generally smaller than the add power of a typical multifocal intraocular lens, which generally have add powers in the range of about 2 Diopters to about 4 Diopters (see, for example, USPN's 6,527,389, 5,225,858, and 6,557,992, which are herein incorporated by reference in their entirety. As used herein, the term “add power” means a change in optical power from an optical power necessary to provide distance vision. As used herein, the “add power” is the change in power at the principal plane of the intraocular lens (e.g., an intraocular lens add power of 4.0 Diopters is approximately equal to an increase in optical power of about 3.2 Diopters in the spectacle lens). Surprisingly, the use of a relatively small add power according to embodiments of the invention (e.g., of about one Diopter to about two Diopters) may beneficially provide better intermediate vision and/or near vision than if a larger add power were to be used in the central zone 314 (e.g., an add power of about 3 or 4 Diopters). This improved performance may, for example, be due to relatively low noise from halo effects when using a lower add power of about 1.0 to about 2.0 Diopters.

Embodiments of the intraocular lens 310 may be configured to provide a pupil 101 dependent visual acuity performance that is preferred over either a spherical intraocular lens such as the intraocular lens 110 or an aspheric intraocular lens such as the intraocular lens 210. For example, both zones 314, 315 in the illustrated embodiment focus light onto or near the retina 104 when the pupil 101 is relatively large, for instance under low lighting conditions or night time driving conditions. Because at least one of the surfaces 312, 313 in the vicinity of the outer zone 315 is aspheric, most of the light from distant objects entering the optic 311 is advantageously focused to substantially a single focus or point. This may provide better visual acuity than is generally possible with an optic having only spherical surfaces (e.g., the intraocular lens 110 illustrated in FIG. 1). The relative areas of the zones 314, 315 may be selected to provide more light energy for distant vision under lower lighting conditions. Thus, while some light from distant object entering the central zone 314 may be slightly defocused, relatively high visual acuity may be maintained, since most of the light entering the optic 311 under these conditions is focused by the outer zone 315 onto the surface of the retina 104

The intraocular lens 310 is also able to provide a pseudo-accommodative benefits under bright or intermediate lighting conditions in which the pupil 101 is small, since under these conditions all or most of the light entering the intraocular lens 310 passes through the central zone 314. Thus, the intraocular lens 310 is able to advantageously provide pseudo-accommodative benefits without significantly compromising the advantages of an aspheric intraocular lens over a spherical intraocular lens during night driving conditions.

The performance of the intraocular lens 310 under differing pupil sizes may be controlled by selecting the diameter of the central zone 314. For example, the central zone 314 may be configured to have an outer diameter D that is about the size of a typical pupil that is fully contracted, such as under sunny outdoor lighting conditions (e.g., the diameter D of the central zone 314 may about 1 millimeter, about 2 millimeters, or about 3 millimeters, or between about 2 millimeters and about 3 millimeters, depending on the relative performance desired between near, intermediate, and distant vision). In other embodiments, the diameter D is selected to provide predetermined areas ratios of the central and outer zones 314, 315 under specific lighting conditions or pupil sizes. Thus, the diameter D may be selected to provide a predetermined performance balance of distant visual acuity and enhanced DOF (or pseudo-accommodation) as a function of pupil size.

In some embodiments, the optic 311 further comprises an intermediate or transition zone 316 disposed between the central and outer zones 314, 315 (optionally indicated by the dashed circle in FIG. 4B). For example, one of the surfaces 312, 313 in the vicinity of the intermediate zone 316 may have a radial profile that is describe by a polynomial and/or spline that may be selected to smoothly blend the at least one of the surfaces of the zones 314, 315. In such embodiments, the diameter of the central zone 314 may not be clearly delineated, in which case the diameter D of the central zone 314 may, for example, be an intermediate diameter between the peripheral region of zone 314 and the inner region of zone 315. Optionally, the intermediate zone 316 may be utilized to further enhance the performance of the intraocular lens 310 in some way. For example, the intermediate zone 316 may be configured to provide better performance when the intraocular lens 310 is decentered or tilted after placement within an eye, as disclosed in U.S. Patent Application Number 2004/0106992, which is herein incorporated by reference in its entirety). Alternatively, the intermediate zone may be used to control halo effects, for example, as disclosed in U.S. Patent Application Number 2006/098163.

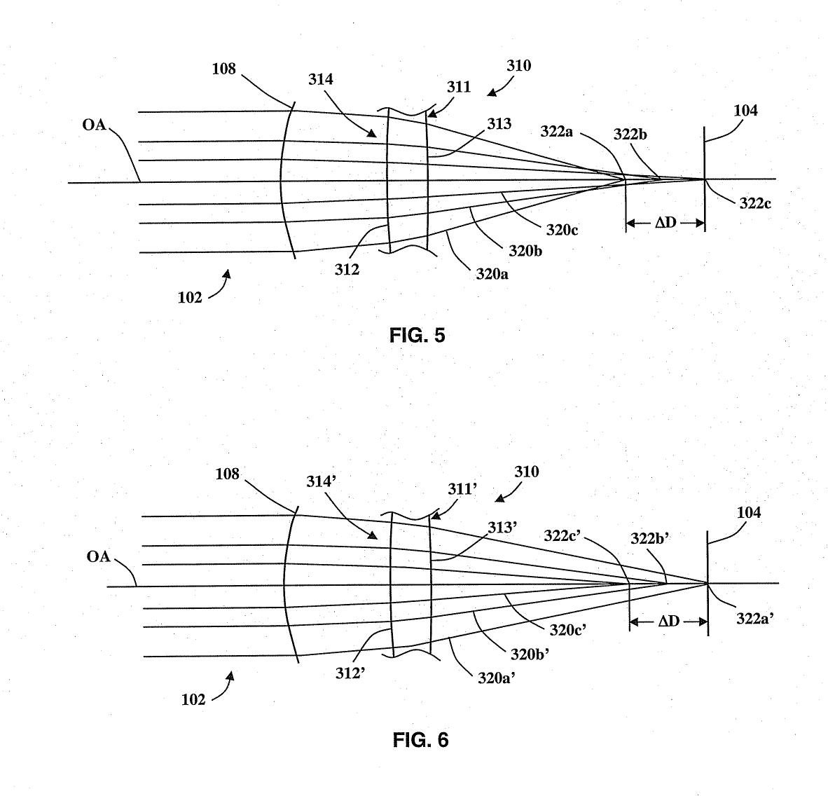

To illustrate one way of configuring the central zone 314 to provide pseudo-accommodation, reference is now made to FIG. 5, which is a magnified side view of the central zone 314 of the optic 311. Three rays 320a, 320b, 320c are shown intercepting the central zone 314 at different radial distances from the optical axis OA. the peripheral ray 320a intercepts a peripheral region of the central zone 314 and is focused along the optical axis OA to a focus 322a, while the paraxial ray 320c intercepts the central zone 314 at or near the optical axis OA and is focused along the optical axis OA to a focus 322c. The intermediate ray 320b intercepts the central zone 314 at a location between the rays 320a, 320c and is focused along the optical axis OA to a focus 322c. In this case the peripheral ray 320a represents a maximum optical power (the first optical power P1) of the central zone 314. The focal length (or optical power) of the central zone 314 may be represented by the distance between the focal point 322b of the intermediate ray 320b and a principle plane of the central zone 314 or the optic 311. Alternatively, the focal length of the central zone 314 may be represented by another point between the foci 322a, 322c.

As illustrated in FIG. 5, the central zone 314 may comprise plurality of optical powers that progressively and continuously increases from the first optical power P1 (e.g., represented by the focus 322c) to the second optical power P2 (e.g., represented by the focus 322a) as the radius from the optical axis OA increases. In such embodiments, the third optical power P3 of the outer zone 315 may be selected to be equal that of first optical power P1 of the central zone 314. Alternatively, the variation in optical power of the central zone 314 with increasing radius from the optical axis OA may have discontinuities and/or may vary in a manner that is not progressive, depending on the particular design requirements or preferences of a designer, practitioner, and/or patient. As discussed in greater detail below, the central zone 314 may alternatively comprise plurality of optical powers that progressively and continuously decreases from a first optical power P1 (e.g., represented by the focus 322c′ in FIG. 6) to the second optical power P2 (e.g., represented by the focus 322a′ in FIG. 6) as the radius from the optical axis OA increases. In any event, the optic 311 is generally configured to provided enhanced visual acuity (e.g., with the outer zone 315) for distant vision and at least reasonably good visual acuity at intermediate distances under certain conditions (e.g., with the central zone 313). In one embodiment, the third optical power P3 is selected to provide distant vision when the intraocular lens is disposed within the eye, while the first and second optical powers P1, P2 are selected so that the central zone 314 provides a visual acuity of at least 20/30, or even 20/20, for objects located at a hyperfocal distance from the eye.

The paraxial ray 320c comes to focus on the retina 104 at the focus 322c, so that objects a optical infinity are just focused and, therefore, at least somewhat resolved by the eye. Referring to the discussion above with regards to FIG. 2, the central zone 314 may be similarly constructed to the optic 211 (e.g., comprising anterior and posterior surfaces that are spherical) so that objects located at the hyperfocal distance advantageously cause the peripheral ray 320a to be focused on the retina 104 and, therefore, to provide a visual acuity of least 20/40, 20/30, or even 20/20. Thus, the central zone 314 in the illustrated embodiment has an extended DOF (represented by the distance ΔD) that provides pseudo-accommodation, by allowing objects located at distances between optical infinity and the hyperfocal distance to resolved by the eye. For object closer than the hyperfocal distance, all the light or rays are generally focused posteriorly to the retina 104, wherein other devices or means may be necessary to provide a desirable level of visual acuity.

In some embodiments, at least one of the surfaces of the central zone 314 has a positive spherical aberration that is greater than or less than that of an equivalent spherical surface having substantially the same focal length or optical power. In general the amount of positive spherical aberration may be selected to provide a predetermined DOF and/or add power, as represented by ΔD in FIG. 5. For example, at least one of the surfaces 312, 313 in the vicinity of the central zone 314 may an oblate surface that has a greater curvature in the periphery than in the center. In such embodiments, the central zone 314 may be configured to suitably resolve objects that are closer than the hyperfocal distance and/or provide enhanced intermediate vision. As used herein the term “intermediate vision” means vision of objects situated approximately 40 centimeters to approximately 1.5 meters from the eye or spectacle plane. By contrast, the term “near vision” means to vision produced by an eye that allows a subject to focus on objects or planes that are relatively close to the subject, for example, within a range of about 25-40 cm or at a distance at which a subject would generally place printed material for the purpose of reading. As used herein, the term “distant vision” means vision produced by an eye that allows a subject to focus on objects or planes that are relatively distant from the subject, preferably at a distance that is greater than about 1 meter to about 2 meters away from the subject, more preferably at a distance of 5 to 6 meters away or greater.

Referring to FIG. 6, the optic 311 may comprise a central zone 314′ that has a negative spherical aberration. For example, in the illustrated embodiment, at least one of the surface 312′, 313′ has a negative spherical aberration that may be selected to produce an overall negative spherical aberration when placed in the eye of a subject. In effect, the optical power of central zone 314′ generally decreases with increasing radial distance from the optical axis OA and may be configured such that the third optical power P3 of the outer zone 315 is equal or substantially equal that of the optical power P2 of the central zone 314. Thus, a peripheral ray 320a′ of the central zone 314′ is focused at or near the retina 104, while intermediate and paraxial rays 320b′, 320c′ are focused progressively closer to the central zone 314′. The resulting add power Δ0D may be represented by the change in focal length over the central zone 314′ (e.g., between the foci 322a′ and 322c′).

In certain embodiments, a method of making an intraocular lens comprises forming an anterior surface and an opposing posterior surface, the surfaces being disposed about an optical axis to provide a clear aperture. The method further comprises forming a central zone comprising a plurality of optical powers that progressively vary between a first optical power at a center of the central zone and a second optical power at a periphery of the central zone, the absolute value of the difference between the first optical power and the second optical power being between about 0.25 Diopter and about 1 Diopter. The method also comprises forming outer zone disposed about the central zone, the outer zone comprising a third optical power and an optional negative spherical aberration. The optic resulting from the method has a variation in optical power over the entire clear aperture that is less than about 1 Diopter.

While embodiments of the invention have been disclosed for an IOL suitable providing enhanced performance under non-optimal conditions, such as when the IOL is decentered from the optical axis of the eye, those skilled in the art will appreciate that embodiments of the invention are suitable for other ocular devices such as contact lenses and corneal implants. For instance, the method of designing a multi-zonal monofocal IOL may be adapted for improving the performance of contact lenses, which are known to move to different positions during use relative to the optical axis of the eye.

What is claimed is:

1. An intraocular lens, comprising:
an optic comprising:
a clear aperture having a diameter;
an anterior surface and an opposing posterior surface, the surfaces disposed about an optical axis;
a central zone having a plurality of optical powers that progressively vary between a first optical power at a center of the central zone and a second optical power at a periphery of the central zone, the absolute value of the difference between the first optical power and the second optical power being between about 0.5 Diopter and about 1.5 Diopters; and
an outer zone disposed about the central zone, the outer zone comprising a third optical power and a negative spherical aberration over the entire zone, the outer zone having an outer diameter that is equal to the diameter of the clear aperture;
the optic having a variation in optical power over the entire clear aperture that is less than about 1.5 Diopters.

2. The intraocular lens of claim 1, wherein the absolute value of the difference between the first optical power and the second optical power is about one Diopter.

3. The intraocular lens of claim 1, wherein the plurality of optical powers progressively and continuously increase from the first optical power to the second optical power.

4. The intraocular lens of claim 1, wherein the plurality of optical powers progressively and continuously increase from the second optical power to the first optical power.

5. The intraocular lens of claim 1, wherein the variation in optical power over the entire clear aperture is less than or equal to about 0.5 Diopter plus the variation in optical power produces by the spherical aberrations of a spherical optic having a nominal optical power that is equal to the third optical power of the outer zone.

6. The intraocular lens of claim 1, wherein negative spherical aberration is selected to at least partially compensate for a spherical aberration of a cornea of the eye.

7. The intraocular lens of claim 1, wherein the anterior and posterior surfaces in the vicinity of the central zone comprises spherical surfaces.

8. The intraocular lens of claim 1, wherein the central zone has a diameter that is between about 1 millimeter and about 3 millimeters.

9. The intraocular lens of claim 1, wherein the first zone and the second zone have at least one surface with a cross-sectional profile described by a polynomial and/or spline.

10. The intraocular lens of claim 1, wherein the zones are configured to provide more light to the retina of the eye for distant vision when light enters the entire central and outer zones.

11. The intraocular lens of claim 1, wherein the first optical power is selected based on the structure of the eye and/or based on a request from a patient.

12. The intraocular lens of claim 1, wherein the negative spherical aberration is selected to at least partially compensate for a spherical aberration of the cornea of the eye.

13. The intraocular lens of claim 1, wherein the negative spherical aberration is selected based on an average ocular aberration of the eyes of a selected population.

14. The intraocular lens of claim 13, wherein the population includes people of a specific age group, people with a cataract, people who have received a corneal ablative procedure, people who are candidates for a corneal ablative procedure, and/or people who are highly myopic or highly hyperopic.

15. The intraocular lens of claim 1, wherein the outer zone has an optical power that is about 20 Diopters and a spherical aberration that is between about −0.19 and about −0.202 microns.

16. The intraocular lens of claim 1, wherein the outer zone has an optical power that is about 20 Diopters and a spherical aberration that is about −0.156 microns.

17. The intraocular lens of claim 1, wherein at least one of the first optical power and the second optical power is equal to the third optical power.

18. The intraocular lens of claim 1, wherein the third optical power is selected to provide distant vision when the intraocular lens is disposed within the eye, and the first optical power and the second optical power are selected so that the central zone provides a visual acuity of at least 20/30, based on the standard Snellen test for visual acuity, for objects located at a hyperfocal distance from the eye.

19. The intraocular lens of claim 1, wherein at least one of the zones has a cylinder power.

20. A method of malting an intraocular lens, comprising:
forming an anterior surface and an opposing posterior surface, the surfaces being disposed about an optical axis and providing a clear aperture;
forming a central zone comprising a plurality of optical powers that progressively vary between a first optical power at a center of the central zone and a second optical power at a periphery of the central zone, the absolute value of the difference between the first optical power and the second optical power being between about 0.25 Diopter and about 1 Diopter;
forming outer zone disposed about the central zone, the outer zone comprising a third optical power and a negative spherical aberration;
the optic having a variation in optical power over the entire clear aperture that is less than about 1 Diopter.
